(19) United States
(12) Patent Application Publication
(10) Pub. No.: US 2008/0161203 A1
(43) Pub. Date: Jul. 3, 2008

(54) MARKERS IDENTIFIED FOR LIVER FIBROSIS AND CIRRHOSIS AND THE MICROARRAY PANEL THEREOF

(76) Inventors: Chun-Lin SU, Wugu Shiang (TW); Ying-Jye Wu, Wugu Shiang (TW)

Correspondence Address: RABIN & Berdo, PC, 1101 14TH STREET, NW, SUITE 500, WASHINGTON, DC, 20005, US

(21) Appl. No.: 11/616,387

(22) Filed: Dec. 27, 2006

(51) Int. Cl. C40B 40/10; C12N 9/50; C07K 14/76; C07K 14/775; C07K 14/00; C12N 9/10; C07K 14/52; C12N 9/02

(52) U.S. Cl. 506/18; 435/219; 530/362; 530/359; 530/350; 435/193; 530/395; 530/356; 530/351; 435/189

(57) ABSTRACT

In the present invention, the markers identified for liver fibrosis and cirrhosis and the microarray panel thereof comprise at least one of the following proteins/genes:

8 up-regulated genes such as
ANXA2; COL1A2; COL3A1; GSN; LDHB; LUM; PDGFRA and TIMP1.
13 down-regulated genes such as
ALB; ANPEP; APOF; AZGP1; BHMT C8A; CFHR4; CFHR5; COL18A1; FTCD; GYS2; ITIH1, and THRAP1.
9 therapeutic targets such as PDGFRA; S100A4; COL3A1; CCL19; DCN; DPT; APP; TNF; and INFG.
The present invention is capable of screening markers for the early warning of the occurrence for severe fibrosis or cirrhosis and potentially targets for drug design.

### BACKGROUND OF THE INVENTION

1. Field of the Invention

The invention relates to a detection of liver damages. More particularly, the invention relates to the markers identified for liver fibrosis and cirrhosis and the microarray panel thereof.

2. Description of Related Art

Many diseases including hepatitis virus infection, alcohol abuse, and long time exposure to organic solvents cause liver damages. The outcomes of liver damages are inflammation, hardening of tissue and even cancer formation. Constant onsets of liver inflammation trigger not only many biochemical events such as immune response, cytokines and chemokines secretion, necrosis, hepatic stellate cells activation and oxidative stress, but also cellular and structural re-organization, namely, fibrosis and cirrhosis (Marcellin et al., Fibrosis and disease progression in hepatitis C, Hepatology 36: S47-S56, 2002).

Fibrosis describes the process in which liver cells recover and patch up wounds; the process is very much alike “scarring” on skin upon wounds. Structurally remodeling is also one of the characteristic phenomena that involve accumulation of extra cellular matrix (ECM) to form scar tissue. Repeated repairs of liver damage result in accumulation of scarring tissue and functional failure of liver cells, such as detoxification and metabolic activities, leading to ultimate severity, that is, cirrhosis. Severe cirrhosis is one of the major causes of death in liver diseases, the other being hepatocellular carcinoma (that is, liver cancer).

Hepatitis C infection alone accounts for estimated 3% of the world population, prevalence followed by hepatitis B infection and alcoholic liver. Continuous hepatitis and fibrosis without proper treatment to intervene disease progress will cause liver to become hardened, swollen and eventually given up operation (decompensation), known as cirrhosis. It took twenty to twenty-five years for hepatitis to develop into severe and irreversible outcome, but often unnoticeable to the patients.

Although current medical protocols suggest, to some extent, fibrosis or cirrhosis may be reversed through the treatment of chronic hepatitis, there's no proper cure to adequately treat fibrosis and cirrhosis. Therapy for liver fibrosis is heavily under development to solve such devastating medical problem. At the mean time, screening tests that can effectively detect liver fibrosis from patients with chronic liver damages are in high demand by physicians.

### SUMMARY OF THE INVENTION

Accordingly, the present invention is directed to the markers identified for liver fibrosis and cirrhosis and the microarray panel thereof which can provide a screening test to identify liver fibrosis from patients with chronic liver damages.

In addition, the present invention is directed to the microarray panel of markers identified for liver fibrosis and cirrhosis, which is capable of screening patients under risk for early warning of the occurrence of severe fibrosis/cirrhosis and potentially targets for drug design.

In the present invention, the markers identified for liver fibrosis and cirrhosis and the microarray panel thereof comprise at least one of the following proteins/genes:

ALB; ANPEP; ANX2; APOF; APP; AZGP1; BHMT; C8A; CCL19; CFHR4; CFHR5; COL1A2; COL3A1; COL18A1; DCN; DPT; FTCD; GYS2; GSN; NFG; LDHB; LUM; ITIH1; PDGFRA; S100A4, THRAP1; TIMP1; and TNF.

Because the 28 proteins/genes can be used in the detection of liver related complications as well as for potential drug target to treat such complications, the present invention can cure or slow down liver fibrosis progression.

Moreover, the test including one or multiple markers from the 28 protein candidates of present invention can be detected by antibody-based methods such as ELISA (Enzyme-Linked Immuno-Sorbent Assay).

### BRIEF DESCRIPTION OF THE DRAWINGS

The application file contains at least one drawing executed in color. Copies of this patent or patent application publication with color drawing(s) will be provided by the Office upon request and payment of the necessary fee.

The accompanying drawings are included to provide a further understanding of the invention, and are incorporated in and constitute a part of this specification. The drawings illustrate embodiments of the invention and, together with the description, serve to explain the principles of the invention.

FIG. 1 shows the table of 28 genes in application for the present invention.

FIG. 2 shows the table of marker panel for screening liver fibrosis.

FIG. 3 shows the table of panel of potential therapeutic targets derived from pathway analysis.

FIG. 4 shows the table of predictive power of marker genes to distinguish the F0/F1 group and F3/F4 group of patients.

FIG. 5 is the hierarchical analysis of differential gene expression according to one of the preferred embodiments of the present invention.

FIG. 6 is the pathway analysis of up-regulated genes in high fibrosis severity liver biopsies according to one of the preferred embodiments of the present invention.

### DETAILED DESCRIPTION OF THE PREFERRED EMBODIMENTS

Reference will now be made in detail to the preferred embodiments of the invention, examples of which are illustrated in the accompanying drawings.

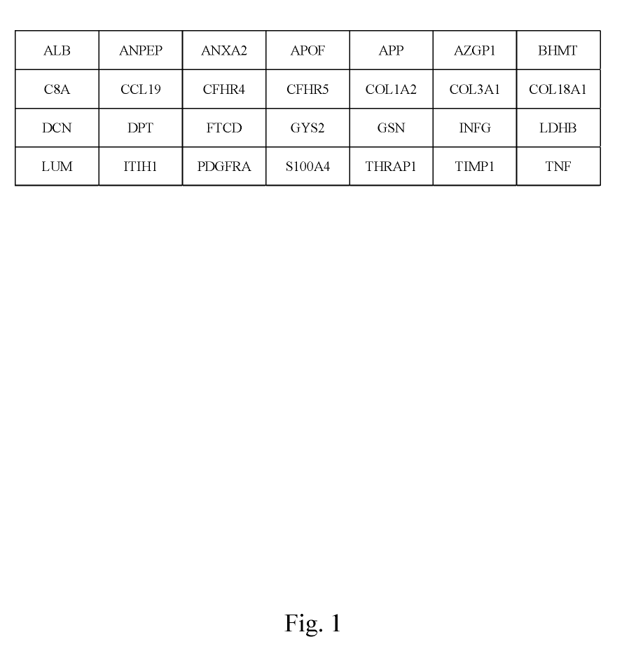

FIG. 1 shows the table of 28 genes in application for the present invention. As shown in FIG. 1, the present invention provides the markers identified for liver fibrosis and cirrhosis and the microarray panel thereof which comprise at least one of the following proteins/genes:

ALB (albumin); ANPEP (alanyl [membrane] aminopeptidase); ANXA2 (annexin A2); APOF (apolipoprotein F); APP (amyloid beta [A4] precursor protein); AZGP1 (alpha-2-glycoprotein 1, zinc-binding); BUT (betaine-homocysteine methyltransferase); C8SA (complement component 8, alpha polypeptide); CCL19 (chemokine [C-C motif] ligand 19); CFHR4 (complement factor H-related 4); CFHR5 (complement factor H-related 5) COL1A2 (collagen, type I, alpha 2); COL3A1 (collagen, type III, alpha 1); COL18A1 (collagen, type XVIII, alpha 1); DCN (decorin); DPT (dermatopontin); FTCD (formiminotransferase cyclodeaminase); GYS2 (glycogen synthase 2); GSN (gelsolin); INFG (interferon gamma) LDHB (lactate dehydrogenase B); LUM (lumican); ITIH1 (inter-alpha [globulin] inhibitor H1); PDGFRA (platelet-derived growth factor receptor, alpha polypeptide); S100A4 (S100 calcium binding protein A4); THRAP1 (thyroid hormone receptor associated protein 1); TIMP1 (TIMP metallopeptidase inhibitor 1); and TNF (tumor necrosis factor).

In the preferred embodiments, at least one of the proteins can be used to differentiate fibrosis stages.

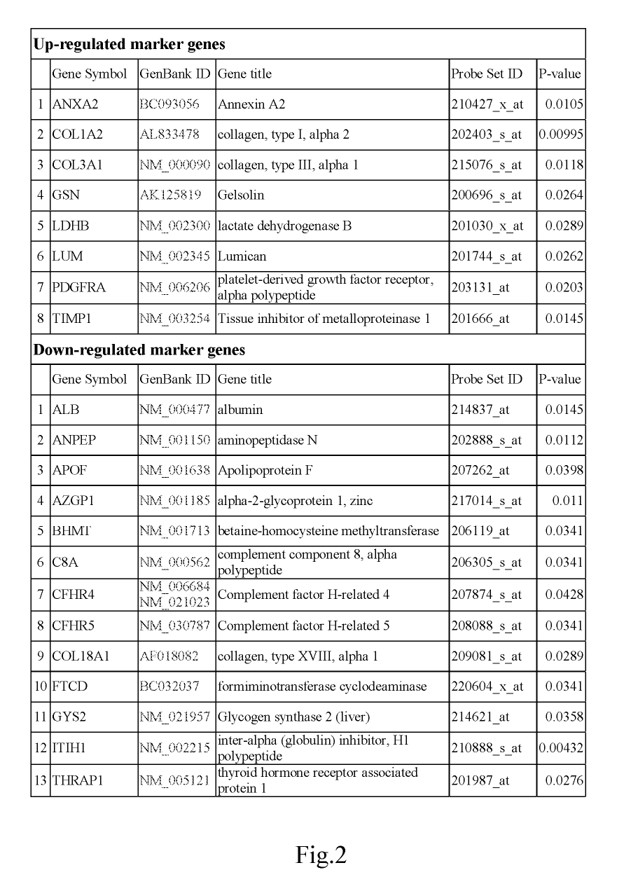

In the preferred embodiments, at least one of the proteins can be served as screening protein markers to detect severe fibrosis or cirrhosis such as the liver samples with the Metavir score F3 or F4. FIG. 2 shows the table of the marker panel for screening liver fibrosis. As shown in FIG. 2, eight of the proteins are up-regulated genes for screening liver fibrosis, such as ANXA2; COL1A2; COL3A1; GSN; LDHB; LUM; PDGFRA; and TIMP1, and thirteen of the proteins are down-regulated genes for screening liver fibrosis, such as ALB; ANPEP; APOF; AZGP1; BHMT; C8A; CFHR4; CFHR5; COL18A1; FTCD; GYS2; ITIH1; and THRAP1.

In the preferred embodiments, at least one of the proteins can be served as indicators for recovery of fibrosis and cirrhosis.

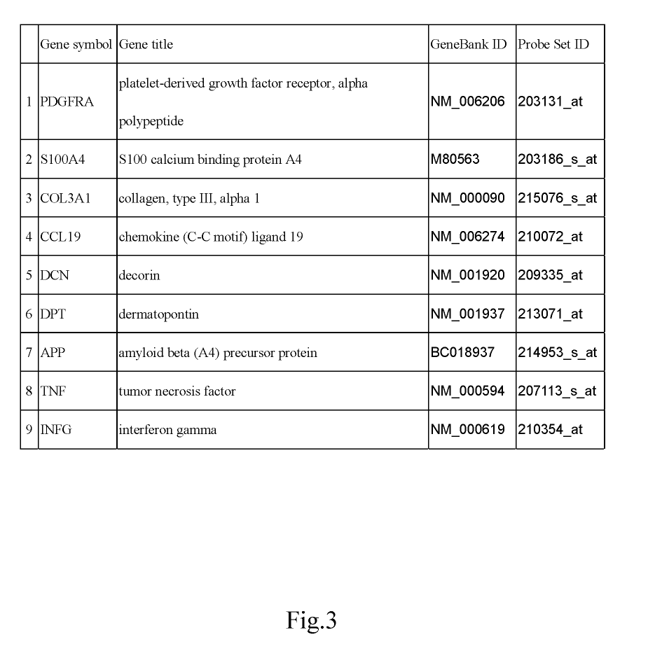

In the preferred embodiments, at least one of the proteins can be used as targets for anti-fibrosis or anti-cirrhosis drug development. FIG. 3 shows the table for the panel of potential therapeutic targets derived from the pathway analysis. As shown in FIG. 3, nine proteins are therapeutic targets, such as PDGFRA; S100A4; COL3A 1; CCL19; DCN; DPT; APP; TNF; and INFG.

In the preferred embodiments, at least one of the proteins can be used in screening methods for compounds that affect the progress of fibrosis or cirrhosis.

In the preferred embodiments, at least one of the proteins can be used in prognostic of liver damages.

In the preferred embodiments, at least one of proteins can be used in an immunodiagnostic kit for identifying liver diseases.

The present invention is resulted from the study of gene expression profiles of liver tissues with progressing fibrosis stages that obtained from chronic hepatitis patients. The panel of differentially expressed genes was identified and can be used to screen markers for the early warning of the occurrence of severe fibrosis/cirrhosis and potentially targets for drug design.

From the up-regulated genes, the present invention constructs a biochemical pathway, which centered by an important gene, TNF (tumor necrosis factor). TNF was linked to the transforming growth factor (TGF) beta-1 and interferon (INF) gamma genes that are also strongly correlated to fibrosis in previous reports. The present invention provides that TNF holds the key to the transition of fibrosis severity and should be a good candidate for curing or slowing down fibrosis progression.

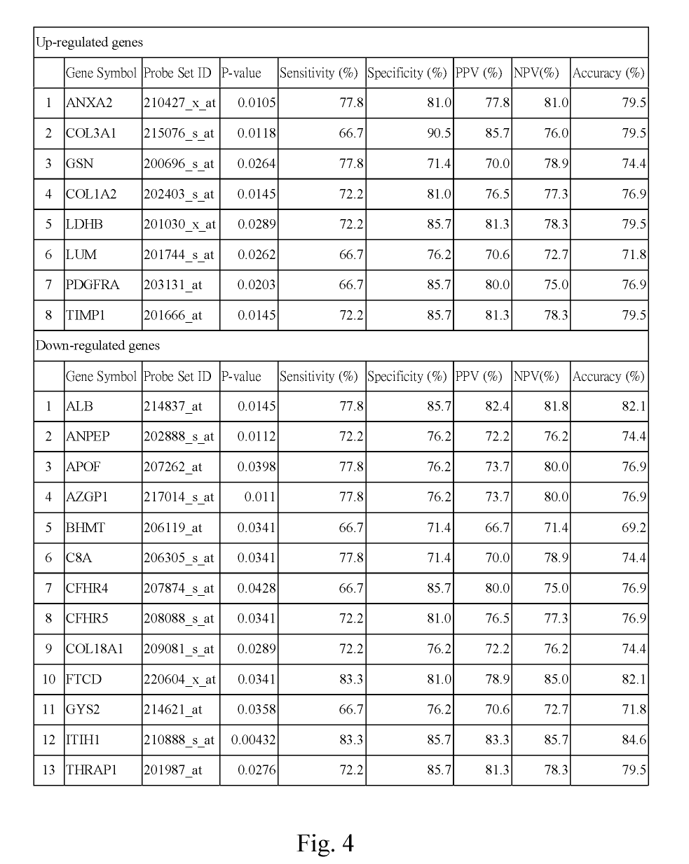

The present invention is the markers identified for liver fibrosis and cirrhosis and the microarray panel thereof, which have varies embodiments with different markers. For a simple and clear statement, a total of 54 liver needle biopsies were collected from chronic hepatitis C patients. FIG. 4 shows the table of predictive power of marker genes to distinguish the F0/F1 group and F3/F4 group of patients, wherein PPV is the positive predictive value and NPV is the negative predictive value. In the following table, the Metavir score was used to classify the fibrosis stages of liver samples and was judged by two independent pathological opinions, inclusion criteria and exclusion criteria, for concordance.

The microarray analysis and patient distribution according to the Metavir scores is as follows.

| Metavir score | F0 | F1 | F2 | F3 | F4 | Accumulation |
|---|---|---|---|---|---|---|
| Patient No. | 10 | 11 | 13 | 10 | 10 | 54 |

### RNA Extraction from Liver Biopsy

Liver biopsies were collected in physician offices and placed into microliter tubes containing RNALater (Ambion, CA, USA) and stored in freezer until use. The TRIZOL method was used to extract RNA from tissues. Basically the reagent (Invitrogen, CA, USA) was added into the tube to mix with tissues and homogenized with plastic pestle followed by phenol extraction (Smith et al., Hepatitis C virus and liver disease; global transcriptional profiling and identification of potential markers, Hepatology 38(6): 1458-67, 2003). Purified RNAs were stored in 70% ethanol and stored in minus 80□ deep freezer. The RNA yield and concentration was detected using spectrophotometer. In average, 5-15 ug total RNA can be purified from a single biopsy about 5 mm in length. RNA quality was assayed by RNA 6000 Nano chip (Aglient, CA, USA) with Bioanalyzer 2100 (Agilent, CA, USA) for detection. The 18S/28S ratio should be higher than 1.2 as intact RNA extract.

### Microarray Experiments

Microarray experiments were performed according to the user manual from manufacturer (Affymetrix, CA, USA). The 2 ug of total RNA from liver tissue was used for the synthesis of first strand cDNA followed by the synthesis of a second strand. Biotinylated cRNA was in vitro synthesized and incubated in fragmentation buffer at 95° C. for 15 minutes. The 1 ug of biotinylated cRNA fragment was then hybridized in a microarray chip with 22,283 probe set, U133A (Affymetrix, CA, USA). Hybridization was conducted in Affymetrix microarray oven (Affymetrix, CA, USA) at 45° C. for 16 hours. After hybridization, the chips were washed and stained in GeneChip® Fluidics Station 400 (Affymetrix, CA, USA) followed by GeneArray Scanner 2500 (Affymetrix, CA, USA) for image acquiring. The quality and raw data was analyzed by Affymetrix microarray suite (version 5.0) software.

### Data Analysis

Results from microarray experiments using Affymetrix human U133A chips were normalized with the Robust Multi-array Average (RMA) method from the BioConductor project (Irizarry et al., Summaries of Affymetrix GeneChip probe level data, Nucleic Acids Res. 31: e15, 2003; http://www.bioconductor.org/) followed by GeneCluster 2.0 (http://www.broad.mit.edu/cancer/software/genecluster2/gc2.html, MA, USA) for process. The GeneSpring software (Silicon Genetics, CA, USA) was used for T test analysis. The Genesis 1.5.0 software was used in the clustering analysis (http://genome.tugraz.at/Software/GenesisCenter.html; Graz University of Technology, Graz, Austria).

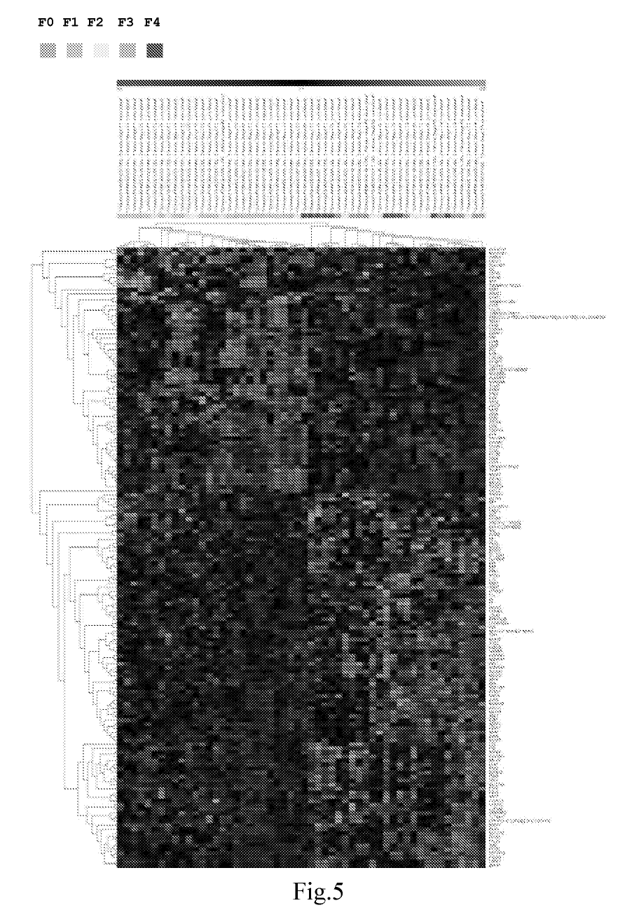

FIG. 5 is the hierarchical analysis of differential gene expression according to one of the preferred embodiments of the present invention. As shown in FIG. 5, the hierarchical analysis of differential gene expression profiles was obtained from the Affymetrix GeneChip U133 experiments for the 54 liver biopsy samples of HCV patients with various stages of liver fibrosis.

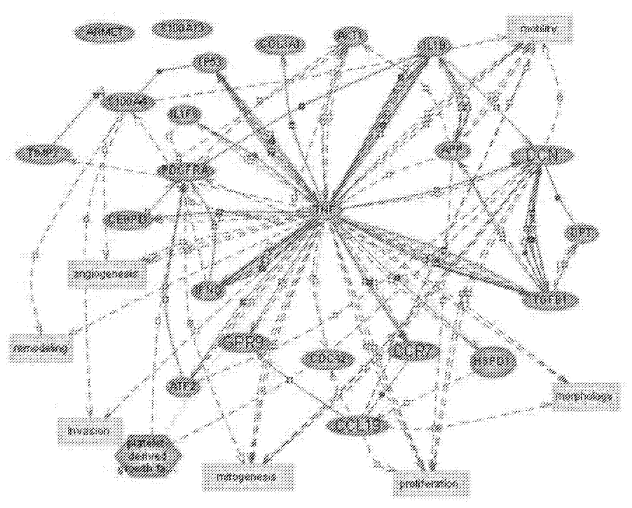

FIG. 6 is the pathway analysis according to one of the preferred embodiments of the present invention. As shown in FIG. 6, the pathway analysis of up-regulated genes was obtained in high fibrosis severity for liver biopsies.

Gene expression in the mRNA level is often related to its presence of protein products. HUPO (Human Proteome Organization, http://www.hupo.org) has done a research project to uncover total proteins that can be detected in human serum or plasma. There were totally 3020 proteins with high fidelity. The present invention provides that, along liver fibrosis progression, there are 8 down-regulated genes and 12 up-regulated genes that can be identified in the HUPO database. These factors can be used in further research to search for serum protein markers that may indicate fibrosis severity.

In the present invention, the markers identified for liver fibrosis and cirrhosis and the microarray panel thereof feature a total number of 28 proteins that can be used in the detection liver related complications as well as for potential drug target to treat such complications.

In one example, TNF (tumor necrosis factor) is used for the development of drugs to cure or slow down liver fibrosis progression.

Patients with chronic hepatitis C infection are in high risk of liver fibrosis. In another example, patient's blood is regularly drawn for a screening test to detect fibrosis turning severe while some current treatments are still available to stop it from worsening. The test includes one or multiple protein markers from the 28 protein candidates can be detected by antibody-based methods such as ELISA (Enzyme-Linked Immuno Sorbent Assay).

In another occasion, patients that are not aware of their liver conditions can be screened for positive in fibrosis and subjected for further diagnosis or treatment.

Yet another example is that hepatitis patients undergone through therapy can be tested by the single or combination of protein markers for their recovery of liver conditions. The markers of the present invention can also serve to test for fibrosis reversion.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. Markers identified for liver fibrosis and cirrhosis comprising at least one of the following proteins/genes:
ALB (albumin); ANPEP (alanyl [membrane] aminopeptidase); ANXA2 (annexin A2); APOF (apolipoprotein F); APP (amyloid beta [A4] precursor protein); AZGP1 (alpha-2-glycoprotein 1, zinc-binding); BHMT (betaine-homocysteine methyltransferase); C8A (complement component 8, alpha polypeptide); CCL19 (chemokine [C-C motif] ligand 19); CFHR4 (complement factor H-related 4); CFHR5 (complement factor H-related 5); COL1A2 (collagen, type I, alpha 2); COL3A1 (collagen, type III, alpha 1); COL18A1 (collagen, type XVIII, alpha 1); DCN (decorin) DPT (dermatopontin); FTCD (formiminotransferase cyclodeaminase); GYS2 (glycogen synthase 2); GSN (gelsolin); INFG (interferon gamma); LDHB (lactate dehydrogenase B); LUM (lumican); ITIH1 (inter-alpha [globulin] inhibitor H1); PDGFRA (platelet-derived growth factor receptor, alpha polypeptide); S100A4 (S100 calcium binding protein A4); THRAP1 (thyroid hormone receptor associated protein 1); TIMP1 (TIMP metallopeptidase inhibitor 1); and TNF (tumor necrosis factor).

2. The markers according to claim 1, wherein at least one of the proteins is used to differentiate fibrosis stages.

3. The markers according to claim 1, wherein at least one of the proteins is served as screening protein markers to detect severe fibrosis or cirrhosis.

4. The markers according to claim 1, wherein eight of the proteins are up-regulated genes for screening liver fibrosis:
ANXA2; COL1A; COL3A1; GSN; LDHB; LUM; PDGFRA; and TIMP1.

5. The markers according to claim 1, wherein thirteen of the proteins are down-regulated genes for screening liver fibrosis:
ALB; ANPEP; APOF; AZGP1; BHMT; C8A; CFHR4; CFHR5; COL18A1; FTCD; GYS2; ITIH1; and THRAP1.

6. The markers according to claim 1, wherein at least one of the proteins is served as indicators for recovery of fibrosis and cirrhosis.

7. The markers according to claim 1, wherein at least one of the proteins is used as targets for anti-fibrosis or anti-cirrhosis drug development.

8. The markers according to claim 1, wherein nine of the proteins are therapeutic targets:
PDGFRA; S100A4; COL3A1; CCL19; DCN; DPT; APP; TNF; and INFG.

9. The markers according to claim 1, wherein at least one of the proteins is used in screening methods for compounds that affect the progress of fibrosis or cirrhosis.

10. The markers according to claim 1, wherein at least one of the proteins is used in prognostic of liver damages.

11. The markers according to claim 1, wherein at least one of proteins is used in an immunodiagnostic kit for identifying liver diseases.

12. A microarray panel of markers identified for liver fibrosis and cirrhosis, comprising at least one of the following proteins/genes:
ALB (albumin); ANPEP (alanyl [membrane] aminopeptidase); ANXA2 (annexin A2); APOF (apolipoprotein F); APP (amyloid beta [A4] precursor protein); AZGP1 (alpha-2-glycoprotein 1, zinc-binding); BHMT (betaine-homocysteine methyltransferase); C8A (complement component 8, alpha polypeptide); CCL19 (chemokine [C-C motif] ligand 19); CFHR4 (complement factor H-related 4); CFHR5 (complement factor H-related 5); COL1A2 (collagen, type I, alpha 2); COL3A1 (collagen, type III, alpha 1); COL18A 1 (collagen, type XVIII, alpha 1); DCN (decorin); DPT (dermatopontin); FTCD (formiminotransferase cyclodeaminase); GYS2 (glycogen synthase 2); GSN (gelsolin); INFG (interferon gamma); LDHB (lactate dehydrogenase B); LUM (lumican); ITIH1 (inter-alpha [globulin] inhibitor H1); PDGFRA (platelet-derived growth factor receptor, alpha polypeptide); S100A4 (S100 calcium binding protein A4); THRAP1 (thyroid hormone receptor associated protein 1); TIMP1 (TIMP metallopeptidase inhibitor 1); and TNF (tumor necrosis factor).

13. The microarray panel according to claim 12, wherein at least one of the proteins is used to differentiate fibrosis stages.

14. The microarray panel according to claim 12, wherein at least one of the proteins is served as screening protein markers to detect severe fibrosis or cirrhosis.

15. The microarray panel according to claim 12, wherein eight of the proteins are up-regulated genes for screening liver fibrosis:
ANXA2, COL1A; COL3A1; GSN; LDHB; LUM; PDGFRA; and TIMP1.

16. The microarray panel according to claim 12, wherein thirteen of the proteins are down-regulated genes for screening liver fibrosis:
ALB; ANPEP; APOF; AZGP1; BHMT; C8A; CFHR4; CFHR5; COL18A1; FTCD, GYS2; ITIH1; and THRAP1.

17. The microarray panel according to claim 12, wherein at least one of the proteins is served as indicators for recovery of fibrosis and cirrhosis.

18. The microarray panel according to claim 12, wherein at least one of the proteins is used as targets for anti-fibrosis or anti-cirrhosis drug development.

19. The microarray panel according to claim 12, wherein nine of the proteins are therapeutic targets:
PDGFRA; S100A4; COL3A1; CCL19; DCN; DPT; APP; TNF; and INFG.

20. The microarray panel according to claim 12, wherein at least one of the proteins is used in screening methods for compounds that affects the progress of fibrosis or cirrhosis.

21. The microarray panel according to claim 12, wherein at least one of the proteins is used in prognostic of liver damages.
